SEQUENCE LISTING (29 sequences)

SEQ ID NO: 1 - DNA, 35 bp, Artificial Sequence
  Sequence of reverse primer G4SGG
tgcgtccgtctctagatccacctcctccagatcca

SEQ ID NO: 2 - DNA, 33 bp, Artificial Sequence
  Sequence of forward primer N11S-4
acgtctctatctgtcttcacactcgaagatttc

SEQ ID NO: 3 - DNA, 35 bp, Artificial Sequence
  Sequence of reverse primer N11S
acgtctctgttatgagttgatggttactcggaaca

SEQ ID NO: 4 - DNA, 34 bp, Artificial Sequence
  Sequence of forward primer amp
tgcgtccgtctccttcgttccactgagcgtcaga

SEQ ID NO: 5 - DNA, 34 bp, Artificial Sequence
  Sequence of reverse primer amp
gctgaccgtctctcgaaaactcacgttaagggat

SEQ ID NO: 6 - DNA, 56 bp, Artificial Sequence
  Sequence of forward primer 3flagGS
tcgtctctgacgataaaggaggtggtggatctggaggaggtggatctgtcttcaca

SEQ ID NO: 7 - DNA, 59 bp, Artificial Sequence
  Sequence of forward primer 3flag oligo
acaaggatgacgacgataaggactataaggacgatgatgacaaggactacaaagatgat

SEQ ID NO: 8 - DNA, 59 bp, Artificial Sequence
  Sequence of reverse primer 3flag oligo
cgtcatcatctttgtagtccttgtcatcatcgtccttatagtccttatcgtcgtcatcc

SEQ ID NO: 9 - DNA, 55 bp, Artificial Sequence
  Sequence of reverse primer 3flagGS
acgtctctttgtaatctgaaccgccaccgcctgatccagacgagagaatctcctc

SEQ ID NO: 10 - DNA, 31 bp, Artificial Sequence
  Sequence of forward primer rop-bsa1
tgtggtctctgaagcgattcacagatgtctg

SEQ ID NO: 11 - DNA, 33 bp, Artificial Sequence
  Sequence of reverse primer rop-bsa1
tgtggtctctcttcacgaccacgctgatgagct

SEQ ID NO: 12 - DNA, 43 bp, Artificial Sequence
  Sequence of forward primer pet28a-Chis2
tgtggtctcttcgggtcaccaccaccaccaccactgagatccg

SEQ ID NO: 13 - DNA, 42 bp, Artificial Sequence
  Sequence of reverse primer pet28a-Chis2
tgtggtctcttcaccatggtatatctccttcttaaagttaaa

SEQ ID NO: 14 - DNA, 37 bp, Artificial Sequence
  Sequence of reverse primer pet28a-Chis3
tgtggtctctatggtatatctccttcttaaagttaaa

SEQ ID NO: 15 - DNA, 45 bp, Artificial Sequence
  Sequence of forward primer pet28a-flag2
tgtggtctctccatggattacaaggatgacgacgataaggtgacc

SEQ ID NO: 16 - DNA, 35 bp, Artificial Sequence
  Sequence of reverse primer pet28a-flag
tgtggtctctccgatgagttgatggttactcggaa

SEQ ID NO: 17 - DNA, 37 bp, Artificial Sequence
  Sequence of forward primer SV40GG2
actcaccgtctcttaactggccgcgactctagatcat

SEQ ID NO: 18 - DNA, 33 bp, Artificial Sequence
  Sequence of forward primer pet28a-cov
tgtggtctctgtgaccggctaccggctgttcga

SEQ ID NO: 19 - DNA, 36 bp, Artificial Sequence
  Sequence of reverse primer pet28a-cov
tgtggtctctccgaatcaacatctggtgatgtatga

SEQ ID NO: 20 - protein, 8 aa, Artificial Sequence
  Sequence of Flag peptide
DYKDDDDK

SEQ ID NO: 21 - protein, 24 aa, Artificial Sequence
  Sequence of Flag peptide litigated by three Flag peptides
DYKDDDDKDYKDDDDKDYKDDDDK

SEQ ID NO: 22 - DNA, 642 bp, Artificial Sequence
  Gene sequence of NanoSwitch-3flag
atggtgaccggctaccggctgttcgaggagattctctcgtctggatcaggcggtggcggttcagattacaaggatgacgacgataaggactataaggacgatgatgacaaggactacaaagatgatgacgataaaggaggtggtggatctggaggaggtggatctgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaactcataa

SEQ ID NO: 23 - DNA, 591 bp, Artificial Sequence
  Gene sequence of NanoSwitch-1flag-2MC
atggtgaccggctaccggctgttcgaggagattctcgactacaaagatgatgacgataaaggaggtggtggatctggaggaggtggatctgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaactcagattacaaggatgacgacgataagtaa

SEQ ID NO: 24 - DNA, 615 bp, Artificial Sequence
  Gene sequence of NanoSwitch-1flag-3
atggattacaaggatgacgacgataaggtgaccggctaccggctgttcgaggagattctcgactacaaagatgatgacgataaaggaggtggtggatctggaggaggtggatctgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaactcagattacaaggatgacgacgataagtaa

SEQ ID NO: 25 - DNA, 591 bp, Artificial Sequence
  Gene sequence of NanoSwitch-1flag-2NM
atggattacaaggatgacgacgataaggtgaccggctaccggctgttcgaggagattctcgactacaaagatgatgacgataaaggaggtggtggatctggaggaggtggatctgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaactcataa

SEQ ID NO: 26 - protein, 25 aa, Artificial Sequence
  poypeptide sequence of pG4
LQPELDSFKEELDKYFKNHTSPDVD

SEQ ID NO: 27 - protein, 18 aa, Artificial Sequence
  poypeptide sequence of p21
PSKPSKRSFIEDLLFNKV

SEQ ID NO: 28 - DNA, 75 bp, Artificial Sequence
  Gene sequence of pG4
ttgcaacctgaattagactcattcaaggaggagttagataaatattttaagaatcatacatcaccagatgttgat

SEQ ID NO: 29 - DNA, 54 bp, Artificial Sequence
  Gene sequence of p21
ccatcaaaaccaagcaagaggtcatttattgaagatctacttttcaacaaagtg

(19) United States
(12) Patent Application Publication
(10) Pub. No.: US 2022/0214342 A1
(43) Pub. Date: Jul. 7, 2022

(54) Protein-based multifunctional molecular switch for antibody detection

(71) Applicant: Chongqing Medical University, Chongqing (CN)

(72) Inventors: Jieli HU, Chongqing (CN); Ailong HUANG, Chongqing (CN); Jie LI, Chongqing (CN)

(21) Appl. No.: 17/568,444

(22) Filed: Jan. 4, 2022

(30) Foreign Application Priority Data
Jan. 5, 2021 (CN) 202110016645.4

(51) Int. Cl. G01N 33/569; C12N 9/02; C07K 19/00

(52) U.S. Cl. CPC G01N 33/569; C07K 19/00; C12N 9/0069

(57) ABSTRACT

The present disclosure discloses a protein-based multifunctional molecular switch for antibody detection, and belongs to the field of protein detection. The molecular switch is a fusion protein including the following parts ligated sequentially from an N-terminus to a C-terminus: a part (1): SmBiT; a part (2): an epitope polypeptide capable of being specifically bound by the antibody to be detected; and a part (3): LgBiT. In the present disclosure, the molecular switch can be specifically recognized by an antibody through the epitope polypeptide, thereby affecting binding of the SmBiT and the LgBiT, and greatly changing a luciferase activity before and after to reflect a concentration level of the antibody. The molecular switch can be used to detect a 2019 novel coronavirus (SARS-CoV-2) with an accuracy and specificity close to 100%, which has an extremely desirable use value.

### CROSS REFERENCE TO RELATED APPLICATION(S)

This patent application claims the benefit and priority of Chinese Patent Application No. 202110016645.4, filed on Jan. 5, 2021, the disclosure of which is incorporated by reference herein in its entirety as part of the present application.

### TECHNICAL FIELD

The present disclosure belongs to the field of protein detection.

### BACKGROUND ART

The protein-based molecular switch is a biosensor that can be used to detect various target biomolecules. Specifically, these sensors respond to the binding of targets by conformational changes and concomitant changes in function. Readouts reflecting these changes can be used to quantify the concentration of the targets. The protein-based molecular switch is widely used in disease diagnosis and basic biomedical research. The latest progress in the design of protein-based molecular switches is represented by the work of David Baker, winner of the 2021 Breakthrough Prize in Life Science. A synthetic protein-based molecular switch LOCKR was designed and constructed (Nature, 2019); the molecular switch was used for biological signal modulation (Nature, 2019), and recently used for antibody and antigen detection of 2019 novel coronavirus (SARS-CoV-2) (Nature, 2021).

Allosteric enzymes are generally used as a basis for designing the molecular switches to detect various protein molecules, including antibodies produced against viruses. Allosteric enzymes here refer to those enzymes with changed activity due to certain conformational changes. When a target molecule is not yet bound to an enzyme molecule, the enzyme molecule has a relatively low (or high) activity; and when the target molecule is bound to the enzyme molecule, a spatial structure of the enzyme molecule is changed, thereby changing the enzyme activity (higher or lower). At this time, the changes in enzyme activity can reflect the existence and quantity of the target molecules. The detection of target molecules by allosteric regulatory enzymes has the advantages as follows: since the binding of the target molecules and the signal output occur on the same molecule, the close coupling of the two helps to ensure a high specificity of the detection, such that many steps such as repeated washing and buffer replacement can be avoided to greatly simplify the detection. By similar principles, fluorescent proteins are also generally used in molecular switch design, with fluorescence instead of enzyme activity as an output signal.

The protein-based molecular switch has been proposed for a long time, but has a relatively desirable detection effect mainly in small molecules; for example, molecular switches are constructed by fluorescent protein, calmodulin (CaM) and calmodulin binding peptide (CaM-BP) to detect calcium ions. After nearly 20 years of iteration and improvement, these molecular switches can now achieve a desirable detection result. Another example is a molecular switch constructed by β-lactamase (BLA) and maltose binding protein (MBP), which can effectively detect maltose. However, compared with these small molecules, the protein-based molecular switches have unsatisfactory results in detecting biological macromolecules. The well-studied β-galactosidase molecular switch is taken as an example. Polypeptides derived from foot-and-mouth disease virus (FMDV) or human immunodeficiency virus (HIV) are recombinantly inserted into specific positions of the β-galactosidase to construct molecular switches, respectively. Even after fully optimized, when conducting serum sample detection using these molecular switches, the signal difference between a patient serum and a control serum does not exceed 5-fold in the best case. The aforementioned David Baker et al. designed and constructed artificial molecular switches using synthetic biology methods, which proposed new ideas on the design basis and path of molecular switches. Although the optimized molecular switch (LOCKR) works well in the detection of an S protein of SARS-CoV-2 (with a signal-to-noise ratio of 17-fold), the LOCKR has a signal-to-noise ratio of only 2-fold when detecting antibodies of SARS-CoV-2 (purified polyclonal antibodies). When detecting antibodies to hepatitis B virus, the LOCKER has a signal-to-noise ratio of at most about 4-fold (testing without serum). These molecular switches may have a lower detection signal-to-noise ratio (a ratio of positive samples to negative samples) in the detection of real clinical samples due to the more complex composition of clinical samples and the more variables and interference factors. In summary, it is difficult for the existing molecular switches to be used in clinical testing due to their relatively low signal-to-noise ratio.

Nanoluc luciferase is isolated from a deep-sea shrimp animal. After modification and optimization, the Nanoluc has the strongest luminescence activity so far (a measured value of a 96-well plate can reach 10⁸ RLU), and has a long half-decay time of luminescence and a small molecular weight. Therefore, Nanoluc is well used in biomedicine. In addition, a 2015 study by Dixon et al. found that a carboxy-terminus fragment with a length of 11 amino acids (AA) in the Nanoluc can be separated from a large amino-terminus fragment (trade name: LgBiT). This 11AA fragment was further modified and evolved to obtain two new peptides, with the trade names of SmBiT and HiBiT (Promega), respectively. There is a high affinity between HiBiT and LgBiT (Kd=0.7 nM), while there is a very low affinity between SmBiT and LgBiT (Kd=190 μM). When the HiBiT or the SmBiT is separated from the LgBiT, each part has no enzymatic activity, while when the HiBiT or the SmBiT is close to the LgBiT, the Nanoluc activity is restored.

To study whether two target proteins interact, Triana et al. fused the two target proteins to the SmBiT and the LgBiT, respectively. When the luciferase activity is activated, it indicates that there is an interaction between the two target proteins. Boursier et al. fused the HiBiT with a G protein-coupled receptor (GPCR) and expressed it on a cell membrane to detect the density of the receptor on a membrane surface; meanwhile, a GPCR ligand and a fluorescent marker each can competitively bind to a HiBiT-GPCR fusion protein to assess the ligand concentration.

### SUMMARY

A problem to be solved by the present disclosure is to provide a novel protein-based multifunctional molecular switch (named NanoSwitch) for antibody detection.

The present disclosure adopts the following technical solutions.

A protein-based multifunctional molecular switch for antibody detection is provided, where the molecular switch is a fusion protein including the following parts ligated sequentially from an N-terminus to a C-terminus:

a part (1): SmBiT;

a part (2): an epitope polypeptide of an antibody to be detected; and

a part (3): LgBiT.

Further, there may be further an epitope polypeptide capable of being specifically bound by the antibody to be detected on an N-terminus side of the part (1) and/or a C-terminus side of the part (3).

Further, there may be a linker sequence between the parts (1) and (2).

Further, the linker sequence may be a flexible linker used for expressing the fusion protein, preferably a Gly-Ser (GS) linker.

Further, the epitope polypeptide may be a Flag tag;

preferably, there may be a linker sequence between the parts (1) and (2);

more preferably, the linker sequence may be a flexible linker used for expressing the fusion protein, furthermore preferably a GS linker.

Further, the epitope polypeptide may be a pG4 polypeptide;

preferably, there may be a linker sequence between the parts (1) and (2);

more preferably, the linker sequence may be a flexible linker used for expressing the fusion protein, furthermore preferably a GS linker.

Further, the epitope polypeptide may be a p21 polypeptide;

preferably, there may be a linker sequence between the parts (1) and (2);

more preferably, the linker sequence may be a flexible linker used for expressing the fusion protein, furthermore preferably a GS linker.

Use of the molecular switch is provided in antibody detection.

A kit for detecting a SARS-CoV-2 includes the molecular switch.

Further, the kit may further include a reagent for detecting a Nanoluc luciferase activity.

FIG. 1 shows a principle of a basic design of the present disclosure. The LgBiT and the SmBiT are ligated through the epitope polypeptide of the antibody to be detected, the SmBiT can enter a specific position of the LgBiT to produce luciferase activity; a target antibody binds to a linear epitope to cause steric and allosteric effects, which affects a binding strength (enhanced or weakened) of the SmBiT and the LgBiT to further change luciferase activity. A luciferase substrate is added, and a content of the target antibody can be reflected through changes in chemiluminescence intensity.

In the present disclosure, a preferred design is to continue to add the epitope polypeptide of the antibody to be detected on the N-terminus side of the SmBiT and/or the C-terminus side of the LgBiT on the basis of FIG. 1. This design can improve the signal-to-noise ratio of detection.

The present disclosure has the following beneficial effects:

1. High signal-to-noise ratio: In the present disclosure, the molecular switch has a signal-to-noise ratio up to above 30-fold in the detection of flag antibodies, and a signal-to-noise ratio up to even above 200-fold in the detection of SARS-CoV-2 antibodies.

2. Desirable specificity: the molecular switch has a detection signal that is not affected by the interference of irrelevant antibodies, and has a specificity of 97.0% in detecting SARS-CoV-2 antibodies.

3. Wide linear range: In the detection of SARS-CoV-2 antibodies, the molecular switch shows desirable linearity when a serum dilution is within 512-fold, which is conducive to quantitative analysis.

4. High accuracy: When detecting the SARS-CoV-2 antibodies, the molecular switch has accuracy up to 97.3%.

Obviously, according to the above-mentioned content of the present disclosure and on the basis of common technical knowledge and common methods in the field, various other modifications, substitutions or alterations can be made without departing from the above-mentioned basic technical idea of the present disclosure.

The above-mentioned content of the present disclosure will be further described in detail below through specific implementations in the form of examples. However, it should not be construed that the subject of the present disclosure is limited to the following examples. Instead, technologies implemented based on the content of the present disclosure should fall within the scope of the present disclosure.

### BRIEF DESCRIPTION OF THE DRAWINGS

FIG. 1 shows a principle of a NanoSwitch molecular switch for antibody detection;

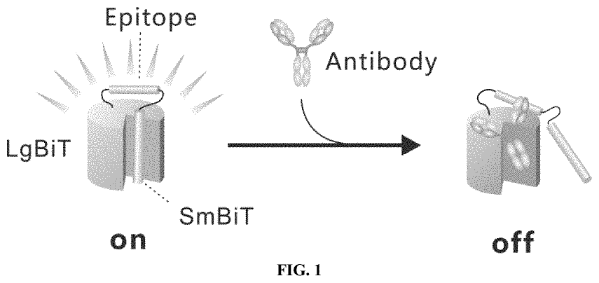

FIG. 2 shows a design and test of the NanoSwitch for Flag antibody detection; where A, structure and working model; B, change in the luciferase activity; C, Interaction between Flag antibody and the NanoSwitch determined by Western blotting;

FIG. 3 shows a test of detection specificity for a NanoSwitch-1×flag-2NM; where A, results of specificity test with a glyceraldehyde-3-phosphate dehydrogenase (GAPDH) antibody; B, results of 3×flag polypeptide competitive inhibition test;

FIG. 4 shows screening of a NanoSwitch for SARS-CoV-2 antibody detection;

FIG. 5 shows results of a polypeptide competitive inhibition experiment of a NanoSwitch-PG4/P21;

FIG. 6 shows a dynamic detection range of a NanoSwitch-pG4;

FIG. 7 shows the NanoSwitch-pG4 in SARS-CoV-2 antibody detection; and

FIG. 8 shows that the NanoSwitch-pG4 correctly reflects the changing trend of the SARS-CoV-2 antibody in patients with corona virus disease 2019 (COVID-19).

### DETAILED DESCRIPTION OF THE EMBODIMENTS

Reagents and material sources used in the examples are shown in Table 1.

TABLE 1
Reagents and material sources

| Reagent name | Manufacturer |
|---|---|
| IPTG | BBI |
| Kanamycin sulfate | BBI |
| Imidazole | BBI |
| NaCl | BBI |
| KCl | BBI |
| Na₂HPO₄ | BBI |
| NaH₂PO₄ | BBI |
| KH₂PO₄ | BBI |
| ATP | NEB |
| DTT | NEB |
| T7 Ligase | NEB |
| BsmB I | Thermo Fisher Scientific |
| 10 × buffer Tango | Thermo Fisher Scientific |
| Lipofectamine 3000 transfection reagent | Thermo Fisher Scientific |
| Fetal bovine serum (FBS) | Gibico |
| Gel extraction kit | Magen |
| PrimeSTAR Max Premix, 2× | TAKARA |
| Plasmid mini extraction kit | OMEGA |
| Agarose | Diamond |
| Yeast extract | Diamond |
| Peptone | Diamond |
| Agar | Diamond |
| Concentrated hydrochloric acid | BBI |
| Sodium hydroxide | BBI |
| Escherichia coli Rosetta (DE3) | Solarbio |
| Prokaryotic expression vector pet28a | Solarbio |
| HEK293 cells | ATCC |

Solutions used in the examples and preparation methods thereof include:

An LB liquid medium: 2 g of tryptone, 1 g of yeast extract and 2 g of NaCl are dissolved in ddH₂O, diluted to 200 ml, and autoclaved for 20 min.

An LB solid medium: 2 g of tryptone, 1 g of yeast extract, 2 g of NaCl and 3 g of agar are dissolved in ddH₂O, diluted to 200 ml, and autoclaved for 20 min.

A kanamycin sulfate solution (50 mg/ml): 0.5 g of kanamycin sulfate solid is dissolved in 10 ml of ddH₂O, filtered and sterilized by a 0.45 μm filter membrane.

A PBS (PH=7.4): 8 g of NaCl, 0.2 g of KCl, 1.78 g of Na₂HPO₄ and 0.24 g of kH₂PO₄ are dissolved in ddH₂O, adjusted to PH=7.4 with concentrated hydrochloric acid, diluted to 1 L, and autoclaved.

A 20 mM phosphate (PH=7.5): 2.39 g of Na₂HPO₄ and 0.38 g of NaH₂PO₄ are dissolved in 1L ddH₂O.

A Binding Buffer (PH=7.5): 29.25 g of NaCl is added to 1 L of the 20 mM phosphate to a final NaCl concentration of 0.5 M, and autoclaved.

A Wash Buffer (PH=7.5): 1.02 g of imidazole is added to 400 mL of the Binding Buffer to a final imidazole concentration of 30 mM, adjusted to PH=7.5 with sodium hydroxide, diluted to 500 mL, and autoclaved.

An Elution Buffer (PH=7.5): 5.1 g of imidazole is added to 200 mL of the Binding Buffer to a final imidazole concentration of 250 mM, adjusted to PH=7.5 with sodium hydroxide, diluted to 300 mL, and autoclaved.

Primer sequences used in the examples are shown in Table 2.

TABLE 2
Primer sequences used in examples of the
present disclosure

| | SEQ | |
|---|---|---|
| Primer | ID | |
| name | NO. | Primer sequence |
| R G4SGG | 1 | TGCGTCCGTCTCTAGATCCACCTCCTCCAGATCCA |
| F N11S-4 | 2 | ACGTCTCTATCTGTCTTCACACTCGAAGATTTC |
| R N11S | 3 | ACGTCTCTGTTATGAGTTGATGGTTACTCGGAACA |
| F amp | 4 | TGCGTCCGTCTCCTTCGTTCCACTGAGCGTCAGA |
| R amp | 5 | GCTGACCGTCTCTCGAAAACTCACGTTAAGGGAT |
| F 3flagGS | 6 | TCGTCTCTGACGATAAAGGAGGTGGTGGATCTGGA |
| | | GGAGGTGGATCTGTCTTCACA |
| F 3flag | 7 | ACAAGGATGACGACGATAAGGACTATAAGGACGAT |
| oligo | | GATGACAAGGACTACAAAGATGAT |
| R 3flag | 8 | CGTCATCATCTTTGTAGTCCTTGTCATCATCGTCC |
| oligo | | TTATAGTCCTTATCGTCGTCATCC |
| R 3flagGS | 9 | ACGTCTCTTTGTAATCTGAACCGCCACCGCCTGAT |
| | | CCAGACGAGAGAATCTCCTC |
| F rop- | 10 | TGTGGTCTCTGAAGCGATTCACAGATGTCTG |
| bsa1 | | |
| R rop- | 11 | TGTGGTCTCTCTTCACGACCACGCTGATGAGCT |
| bsa1 | | |
| F pet28a- | 12 | TGTGGTCTCTTCGGGTCACCACCACCACCACCAC |
| Chis2 | | TGAGATCCG |
| R pet28a- | 13 | TGTGGTCTCTTCACCATGGTATATCTCCTTCTTA |
| Chis2 | | AAGTTAAA |
| R pet28a- | 14 | TGTGGTCTCTATGGTATATCTCCTTCTTAAAGTT |
| Chis3 | | AAA |
| F pet28a- | 15 | TGTGGTCTCTCCATGGATTACAAGGATGACGACG |
| flag2 | | ATAAGGTGACC |
| R pet28a- | 16 | TGTGGTCTCTCCGATGAGTTGATGGTTACTCGGA |
| flag | | A |
| F SV40GG2 | 17 | ACTCACCGTCTCTTAACTGGCCGCGACTCTAGAT |
| | | CAT |
| F pet28a- | 18 | TGTGGTCTCTGTGACCGGCTACCGGCTGTTCGA |
| cov | | |
| R pet28a- | 19 | TGTGGTCTCTCCGAATCAACATCTGGTGATGTA |
| cov | | TGA |

### Example 1 A NanoSwitch Molecular Switch for Flag Antibody Detection

I. Principle

In this example, a Flag antibody was taken as an example to demonstrate a basic design of the present disclosure. The design ideas and working principles were as follows:

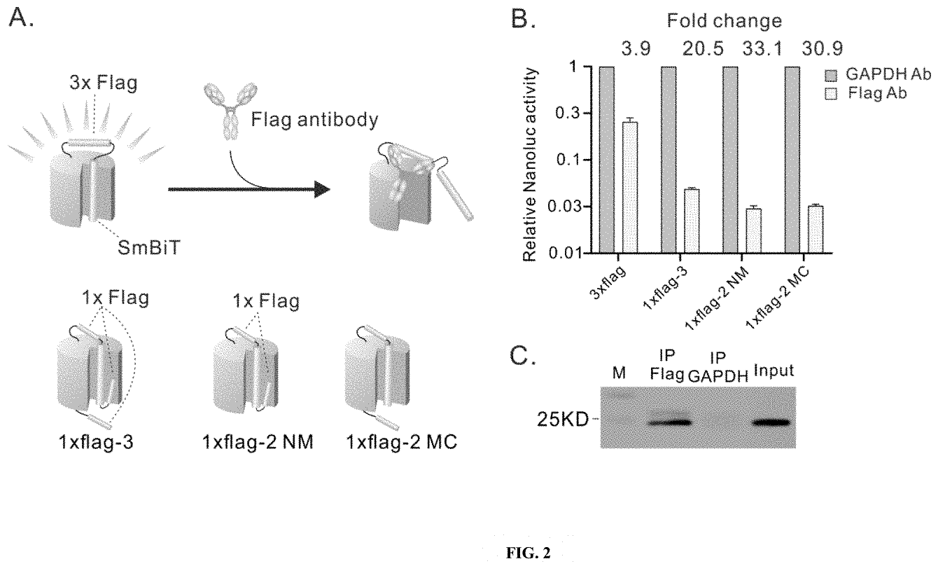

(1) SmBiT was fused to a N-terminus of LgBiT; (2) a polypeptide that can bind to an antibody, such as 3×flag, was inserted between the LgBiT and the SmBiT (FIG. 2A); (3) when there was no antibody, SmBiT was able to bind to a corresponding position of the LgBiT, and NanoSwitch was in a fully active state; (4) when a specific antibody existed and was bound to a specific peptide in the NanoSwitch, steric hindrance and allosteric effects were caused, leading to binding obstacles between the SmBiT and the LgBiT. At this time, the NanoSwitch was in a partially inactive state, showing that the signal decreased after the substrate was added (FIG. 2A). Therefore, this signal change can reflect the amount of specific antibody molecules.

Flag, also known as a “Flag tag”, is a common artificial tag polypeptide for detecting overexpressed proteins, and has a sequence of: DYKDDDDK (SEQ ID NO. 20); the Flag has a common replacement form 3×Flag, which is obtained by ligating another two Flag tags on the Flag, namely DYKDDDDK DYKDDDDK DYKDDDDK (SEQ ID NO. 21).

II. Method

1. Construction of SmBiT-LgBiT Plasmid

The plasmid SmBiT-LgBiT was constructed in two steps; a transitional plasmid SmBiT-HBC was constructed, where a construction process was as follows: using a plasmid RlucN-HBC (same as a plasmid RlucN-HBC in Chinese patent CN201610564291.6) as a template, amplification was conducted using primers F G4SGG7+R bsmb1vect; a PCR reaction system was: 10 ng of the plasmid RlucN-HBC, 0.4 μl for each of the primers F G4SGG7 (10 μM) and R amp (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 1 min, conducting 35 cycles. An amplified fragment was recovered using the gel extraction kit, and the recovered fragment was named as frag1.

The oligonucleotides F C11 oligo and R C11 oligo were annealed and 5′-phosphorylated. A reaction system was 10 including: 1 μl of F C11 oligo (100 μM), 1 μl of R C11 oligo (100 μM), 1 μl of a 10X T4 ligase buffer, 0.5 μl of a T4 polynucleotide kinase and 6.5 μl of ddH₂O. A reaction tube was placed on a PCR machine for reaction at 37° C. for 30 min and at 95° C. for 5 min, and then into a cooling cycle; each cycle was reduced by 1° C. for 15 sec; after 70 cycles, the temperature was reduced to 25° C. to terminate the reaction. 1 μl of a reaction product was diluted with 199 μl of ddH₂O, and a diluted product (named frag2) and the frag1 were subjected to Golden Gate ligation reaction. A reaction system was: 0.75 μl of BsmB I enzyme, 1 μl of Tango buffer, 1 μl of DTT, 0.25 μl of T7 DNA ligase, 1 μl of ATP, 1 μl of frag3 (100 ng), 1 μl of frag4, and ddH₂O was added to make up to 10μl. Reaction was conducted by: 37° C. for 4 min, 20° C. for 4 min, conducting 20 cycles. Inactivation reaction was conducted at 80° C. for 20 min. A Golden Gate product was transformed into JM109 competent bacteria, the bacteria were coated on plates, clones were screened, and sequenced for identification; a correct clone was named as a plasmid SmBiT-HBC.

A plasmid pNanoluc (synthesized and cloned by Tsingke Biotechnology Co., Ltd., for the Nanoluc luciferase gene sequence, referring to: Dixson et al., NanoLuc Complementation Reporter Optimized for Accurate Measurement of Protein Interactions in Cells. ACS Chemical Biology, 2015) was used as a template, and amplification was conducted with primers F N11S-4+R N11 S. A PCR reaction system was: 10 ng of plasmid Nanoluc, 0.4 μl for each of primers F N11S-4 (10 μM) and R N11S (10 μM), 10 μl of 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 20 sec, conducting 35 cycles. An amplified fragment was recovered using the gel extraction kit, and the recovered fragment was named as frag3. The plasmid SmBiT-HBC was used as a template, and amplification was conducted using primers F SV40GG2+R amp; a PCR reaction system was: 10 ng of the plasmid SmBiT-HBC, 0.4 μl for each of the primers F SV40GG2 (10 μM) and R amp (10 μM), 10 μl of a 2× PrimeSTAR Max Premix, and sterilized ultrapure water was added to make up a volume of 20 μl. The amplification reaction was conducted by: pre-denaturation at 95° C. for 3 min; denaturation at 95° C. for 15 sec, at 55° C. for 15 sec and at 72° C. for 1 min, conducting 35 cycles. An amplified fragment was recovered using the gel extraction kit, and the recovered fragment was named as frag4. The plasmid SmBiT-HBC was used as a template, and amplification was conducted using primers F amp+R G4SGG; a PCR reaction system was: 10 ng of the plasmid SmBiT-HBC, 0.4 μl for each of the primers F amp (10 μM) and R G4SGG (10 μM), 100 of a 2x PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 1 min, conducting 35 cycles. An amplified fragment was recovered using the gel extraction kit, and the recovered fragment was named as frag5.

The three fragments frag 3, fra4 and frag5 obtained above were subjected to Golden Gate ligation reaction. A reaction system was: 0.75 μl of BsmB I enzyme, 1 μl of Tango buffer, 1 μl of DTT, 0.25 μl of T7 DNA ligase, 1 μl of ATP, 1 μl of frag3 (15 ng), 2 μl of frag4 (60 ng), 1 μl of frag5 (60 ng), and ddH2O was added to make up to 10 μl. Reaction was conducted by: 37° C. for 4 min, 20° C. for 4 min, conducting 20 cycles. Inactivation reaction was conducted at 80° C. for 20 min. A Golden Gate product was transformed into JM109 competent bacteria. The bacteria were coated on plates, clones were screened, and sequenced for identification; a correct clone was named as a plasmid SmBiT-LgBiT.

2. Construction of a Plasmid NanoSwitch-3×Flag

The plasmid SmBiT-LgBiT was used as a template and amplification was conducted using primers F 3flag GS+R amp. A PCR reaction system was: 10 ng of the plasmid SmBiT-LgBiT, 0.4 μl for each of the primers F 3flag GS (10 μM) and R amp (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 30 sec, conducting 35 cycles. An amplified fragment was recovered using the gel extraction kit, and the recovered fragment was named as frag6.

The plasmid SmBiT-LgBiT was used as a template and amplification was conducted using primers R 3flag GS+F amp. A PCR reaction system was: 10 ng of the plasmid SmBiT-LgBiT, 0.4 μl for each of the primers R 3flag GS (10 μM) and F amp (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 30 sec, conducting 35 cycles. An amplified fragment was recovered using the gel extraction kit, and the recovered fragment was named as frag7.

The oligonucleotides F 3flag oligo and R 3flag oligo were annealed and 5′-phosphorylated. A reaction system was 10 including: 1 μl of F 3flag oligo (100 μM), 1 μl of R 3flag oligo (100 μM), 1 μl of a 10X T4 ligase buffer, 0.5 μl of a T4 polynucleotide kinase, and 6.5 μl of ddH₂O. A reaction tube was placed on a PCR machine for reaction at 37° C. for 30 min at 95° C. for 5 min, and then into a cooling cycle; each cycle was reduced by 1° C. for 15 sec; after 70 cycles, the temperature was reduced to 25° C. to terminate the reaction. 1 μl of a reaction product was diluted with 199 μl of ddH₂O, and the fragment was named as frag8.

The three fragments frag6, frag7 and frag8 obtained above were subjected to the Golden Gate ligation reaction. A reaction system was: 0.75 μl of BsmB I enzyme, 1 μl of Tango buffer, 1 μl of DTT, 0.25 μl of T7 DNA ligase, 1 μl of ATP, 1 μl of frag6 (50 ng), 1 μl of frag7 (50 ng), 1μl of frag8, and ddH₂O was added to make up to 10 μl. Reaction was conducted by: 37° C. for 4 min, 20° C. for 4 min, conducting 20 cycles. Inactivation reaction was conducted at 80° C. for 20 min. A Golden Gate product was transformed into JM109 competent bacteria., The bacteria were coated on plates, clones were screened, and sequenced for identification; a correct clone was named as a plasmid NanoSwitch-3×flag.

The NanoSwitch-3×flag has a molecular switch gene sequence (SEQ ID NO. 22) as follows:

ATGGTGACCGGCTACCGGCTGTTCGAGGAGATTCTCtcgtctggatcaggcggtggcggttca
GATTACAAGGATGACGACGATAAGGACTATAAGGACGATGATGACAAGGACTACAAA
GATGATGACGATAAAggaggtggtggatctggaggaggtggatct

[symbol]

[symbol]

[symbol]

[symbol]

[symbol]

[symbol]

[symbol]

[symbol]

[symbol]

[symbol]

TAA

Note: The first 3 bases and the end 3 bases of the sequence are a start codon and a stop codon, respectively; the unbold part with single underline is SmBiT, and a lower case part is a linker sequence GS linker; a part with double underline is 3×flag, and the bold part with single underline is LgBiT. GS linker is a polypeptide composed of amino acids G and S, has many permutations and combinations, and can routinely replace a linker sequence of the NanoSwitch-3×flag molecular switch.

3. Flag Antibody Test

The plasmid NanoSwitch-3×flag was transfected into HEK293 cells; after 48 h, the cells were lysed by repeated freezing and thawing in liquid nitrogen, and 1 μl of flag antibody (1 μg) was added to 9 μl of lysate (1 μg of GAPDH antibody was added to a control group); after incubation for 1 h at 37° C., the Nanoluc activity was detected using a commercially available Nanoluc detection reagent (Promega).

### III. Results

The results show that compared with the control group, the flag antibody reduces the signal by an average of 3.9-fold (FIG. 2B).

The results in this example show that the molecular switch of the present disclosure can effectively achieve antibody detection.

### Example 2 Improvement of a NanoSwitch-3×flag

In this example, three improvement schemes were given on the basis of Example 1.

Improvement scheme 1: three 1×flags were placed on an N-terminus of SmBiT, between the SmBiT and LgBiT, and a C-terminus of the LgBiT (NanoSwitch-1×flag-3).

Improvement scheme 2, on the basis of improvement scheme 1, only the first two 1×flag were retained, a 1×flag at the C-terminus was removed (NanoSwitch-1×flag-2NM).

Improvement scheme 3, on the basis of improvement scheme 1, only the last two 1×flag were retained, a 1×flag at the N-terminus was removed (NanoSwitch-1×flag-2MC).

### I. Method

1. Construction of a Plasmid NanoSwitch-1×flag-2MC

The plasmid NanoSwitch-3×flag was used as a template and amplification was conducted using primers F flag-SV40 +R amp. A PCR reaction system was: 10 ng of the plasmid C11-3flag-N11S, 0.4 μl for each of the primers F flag-SV40 (10 μM) and R amp (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 30 sec, conducting 35 cycles. An amplified fragment was recovered using the gel extraction kit, and the recovered fragment was named as frag9.

The plasmid NanoSwitch-3×flag was used as a template and amplification was conducted using primers R flag-N11S+F C11-FLAG. A PCR reaction system was: 10 ng of the plasmid C11-3flag-N11S, 0.4 μl for each of the primers R flag-N11S (10 μM) and F C11-FLAG (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 20 sec, conducting 35 cycles. An amplified fragment was recovered using the gel extraction kit, and the recovered fragment was named as frag10.

The plasmid NanoSwitch-3×flag was used as a template and amplification was conducted using primers F amp +R C11-FLAG. A PCR reaction system was: 10 ng of the plasmid C11-3flag-N11S, 0.4 μl for each of the primers F amp (10 μM) and R C11-FLAG (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 30 sec, conducting 35 cycles. An amplified fragment was recovered using the gel extraction kit, and the recovered fragment was named as frag11.

The three fragments frag9, frag10 and frag11 obtained above were subjected to Golden Gate ligation reaction. A reaction system was: 0.75 μl of BsmB I enzyme, 1 μl of Tango buffer, 1 μl of DTT, 0.25 μl of T7 DNA ligase, 1 μl of ATP, 1 μl of frag9 (40 ng), 1 μl of frag10 (10 ng), 1 μl of frag11 (40 ng), and ddH2O was added to make up to 10 μl. Reaction was conducted by: 37° C. for 4 min, 20° C. for 4 min, conducting 20 cycles. Inactivation reaction was conducted at 80° C. for 20 min. A Golden Gate product was transformed into JM109 competent bacteria, the bacteria were coated on plates, clones were screened, and sequenced for identification; a correct clone was named as a plasmid NanoSwitch-1×flag-2MC.

The NanoSwitch-1×flag-2MC has a molecular switch gene sequence (SEQ ID NO. 23) as follows:

ATGGTGACCGGCTACCGGCTGTTCGAGGAGATTCTCGACTACAAAGATGATGA
CGATAAAggaggtggtggatctggaggaggtggatct

[symbol]

[symbol]

[symbol]

[symbol]

[symbol]

[symbol]

[symbol]

[symbol]

[symbol]

[symbol]

GATTACAAGGATGACGACGATAAGTAA

Note: The first 3 bases and the end 3 bases of the sequence are a start codon and a stop codon, respectively; the unbold part with single underline is SmBiT, and a lower case part is a linker sequence (GS linker); a part with double underline is 1×flag, and the bold part with single underline is LgBiT.

2. NanoSwitch-1×flag-3

The plasmid NanoSwitch-1×flag-2MC was used as a template and amplification was conducted using primers F flag-C11 +R amp. A PCR reaction system was: 10 ng of the plasmid C11-noGS-flag-10GS-N11S-noGS-flag, 0.4 μl for each of the primers F flag-C11 (10 μM) and R amp (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 30 sec, conducting 35 cycles. An amplified fragment was recovered using the gel extraction kit, and the recovered fragment was named as frag12.

The plasmid NanoSwitch-1×flag-2MC was used as a template and amplification was conducted using primers R flag-C11+F amp. A PCR reaction system was: 10 ng of the plasmid C11-noGS-flag-10GS-N11S-noGS-flag, 0.4 μl for each of the primers R flag-C11 (10 μM) and F amp (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 30 sec, conducting 35 cycles. An amplified fragment was recovered using the gel extraction kit, and the recovered fragment was named as frag13.

The two fragments frag12 and frag13 obtained above were subjected to Golden Gate ligation reaction. A reaction system was: 0.75 μl of BsmB I enzyme, 1 μl of Tango buffer, 1 μl of DTT, 0.25 μl of T7 DNA ligase, 1 μl of ATP, 1 μl of frag12 (50 ng), 1 μl of frag13 (40 ng), and ddH₂O was added to make up to 10 μl. Reaction was conducted by: 37° C. for 4 min, 20° C. for 4 min, conducting 20 cycles. Inactivation reaction was conducted at 80° C. for 20 min. A Golden Gate product was transformed into JM109 competent bacteria, the bacteria were coated on plates, clones were screened, and sequenced for identification; a correct clone was named as a plasmid NanoSwitch-1×flag-3.

The NanoSwitch-1×flag-3 has a molecular switch gene sequence (SEQ ID NO. 24) as follows:

ATGGATTACAAGGATGACGACGATAAGGTGACCGGCTACCGGCTGTTCGAGG
AGATTCTCGACTACAAAGATGATGACGATAAAggaggtggtggatctggaggaggtggatct

[symbol]

[symbol]

[symbol]

[symbol]

[symbol]

[symbol]

[symbol]

[symbol]

[symbol]

[symbol]

GATTACAAGGATGACGA
CGATAAGTAA

Note: The first 3 bases and the end 3 bases of the sequence are a start codon and a stop codon, respectively; the unbold part with single underline is SmBiT, and a lower case part is a linker sequence (GS linker); a part with double underline is 3×flag, and the bold part with single underline is LgBiT.

3. NanoSwitch-1×flag-2NM

The plasmid NanoSwitch-1×flag-3 was used as a template and amplification was conducted using primers F SV40 GG2 +R amp. A PCR reaction system was: 10 ng of the plasmid Flag-noGS-C11-noGS-flag-10GS-N11S-noGS-flag, 0.4 μl for each of the primers F SV40 GG2 (10 μM) and R amp (10 μM), 10 μl of a 2× PrimeSTAR Max Premix, and sterilized ultrapure water was added to make up a volume to 20 The amplification reaction was conducted by: pre-denaturation at 95° C. for 3 min; denaturation at 95° C. for 15 sec, at 55° C. for 15 sec and at 72° C. for 30 sec, conducting 35 cycles. An amplified fragment was recovered using the gel extraction kit, and the recovered fragment was named as frag14.

The plasmid NanoSwitch-1×flag-3 was used as a template and amplification was conducted using primers R N11S-del+F amp. A PCR reaction system was: 10 ng of the plasmid Flag-noGS-C11-noGS-flag-10GS-N11S-noGS-flag, 0.4 μl for each of the primers RN11S-del (10 μM) and F amp (10 μM), 10 μl of a 2× PrimeSTAR Max Premix, and sterilized ultrapure water was added to make up a volume to 20 The amplification reaction was conducted by: pre-denaturation at 95° C. for 3 min; denaturation at 95° C. for 15 sec, at 55° C. for 15 sec and at 72° C. for 30 sec, conducting 35 cycles. An amplified fragment was recovered using the gel extraction kit, and the recovered fragment was named as frag15.

The two fragments frag14 and frag15 obtained above were subjected to Golden Gate ligation reaction. A reaction system was: 0.75 μl of BsmB I enzyme, 1 μl of Tango buffer, 1 μl of DTT, 0.25 μl of T7 DNA ligase, 1 μl of ATP, 1 μl of frag14 (50 ng), 1 μl of frag15 (30 ng), and ddH₂O was added to make up to 10 μl. Reaction was conducted by: 37° C. for 4 min, 20° C. for 4 min, conducting 20 cycles. Inactivation reaction was conducted at 80° C. for 20 min. A Golden Gate product was transformed into JM109 competent bacteria, the bacteria were coated on plates, clones were screened, and sequenced for identification; a correct clone was named as a plasmid NanoSwitch-1×flag-2NM.

The NanoSwitch-1×flag-2NM has a molecular switch gene sequence (SEQ ID NO. 25) as follows:

ATGGATTACAAGGATGACGACGATAAGGTGACCGGCTACCGGCTGTTCGAGG
AGATTCTCGACTACAAAGATGATGACGATAAAggaggtggtggatctggaggaggtggatct

[symbol]

[symbol]

[symbol]

[symbol]

[symbol]

[symbol]

[symbol]

[symbol]

[symbol]

[symbol]

TAA

Note: The first 3 bases and the end 3 bases of the sequence are a start codon and a stop codon, respectively; the unbold part with single underline is SmBiT, and a lower case part is a linker sequence (GS linker); a part with double underline is 3×flag, and the bold part with single underline is LgBiT.

4. Flag Antibody Detection

The constructed expression plasmids were transfected into HEK293 cells, respectively; 1 μl of flag antibody (1 μg) was added to 9 μl of lysate (1 μg of GAPDH antibody was added to a control group); after incubation for 1 h at 37° C., the luciferase activity was detected.

5. Affinity Purification Assay

To further prove that the flag antibody can indeed bind to the NanoSwitch with the flag polypeptide, the flag antibody or the GAPDH antibody were incubated with the lysate of HEK293 cells expressing NanoSwitch-1×flag-2NM, respectively; and affinity purification was conducted using Protein A Sepharose beads, and a resulting product was subjected to Western blot identification.

II. Results

1. Flag Antibody Detection

Compared with the control group, the flag antibody reduces the NanoSwitch-1×flag-3 signal by an average of 20.5-fold, reduces the NanoSwitch-1×flag-2NM signal by an average of 33.1-fold, and reduces the NanoSwitch-1×flag-2MC signal by an average of 30.9-fold (FIG. 2B).

2. Affinity Purification Assay

The flag antibody can precipitate NanoSwitch-1×flag-2NM, while the GAPDH antibody cannot (FIG. 2C). It indicates that the flag antibody can indeed bind to the NanoSwitch-1×flag-2NM molecule in a natural state.

The results of this example show that compared with the basic design of Example 1, the three improvement schemes significantly improve the detection signal-to-noise ratio.

### Example 3 Specificity of NanoSwitch-1×flag-2 in Flag Antibody Detection

Prokaryotic expression and purification were conducted for NanoSwitch-1×flag-2NM to further prove a detection specificity of flag antibody.

I. Method

1. Plasmid Construction and Prokaryotic Expression and Purification

The plasmid PET28a was used as a template and amplification was conducted using primers F pet28a-Chis2+R rop-bsa1. A PCR reaction system was: 10 ng of the plasmid PET28a, 0.4 μl for each of the primers F pet28a-Chis2 (10 μM) and R rop-bsa1 (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 45 sec, conducting 35 cycles. An amplified fragment was recovered using the gel extraction kit, and the recovered fragment was named as frag16.

The plasmid PET28a was used as a template and amplification was conducted using primers R pet28a-Chis3+F rop-bsa1. A PCR reaction system was: 10 ng of the plasmid PET28a, 0.4 μl for each of the primers R pet28a-Chis3 (10 μM) and F rop-bsa1 (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 45 sec, conducting 35 cycles. An amplified fragment was recovered using the gel extraction kit, and the recovered fragment was named as frag17.

The plasmid NanoSwitch-1×flag-2 was used as a template and amplification was conducted using primers F pet28a-flag2+R pet28a-flag. A PCR reaction system was: 10 ng of the plasmid NanoSwitch-1×flag-2, 0.4 μl for each of the primers F pet28a-flag2 (10 μM) and R pet28a-flag (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 20 sec, conducting 35 cycles. An amplified fragment was recovered using the gel extraction kit, and the recovered fragment was named as frag18.

The three fragments frag16, frag17 and frag18 obtained above were subjected to Golden Gate ligation reaction. A reaction system was: 0.75 μl of BsmB I enzyme, 1 μl of Tango buffer, 1 μl of DTT, 0.25 μl of T7 DNA ligase, 1 μl of ATP, 1 μl of frag16 (60 ng), 1 μl of frag17 (60 ng), 1 μl of frag18 (15 ng), and ddH2O was added to make up to 10 μl. Reaction was conducted by: 37° C. for 4 min, 20° C. for 4 min, conducting 20 cycles. Inactivation reaction was conducted at 80° C. for 20 min. A Golden Gate product was transformed into JM109 competent bacteria, the bacteria were coated on plates, clones were screened, and sequenced for identification; a correct clone was named as a plasmid PET-NanoSwitch-1×flag-2.

The plasmid PET-NanoSwitch-1×flag-2 was transformed into Rosetta (DE3), spread on an LB plate containing 50 μg/ml kanamycin sulfate, and incubated at 37° C. for 16 h. A single colony was incubated in a 5 ml LB medium containing 50 μg/ml kanamycin sulfate, at 37° C., 220 rpm for 16 h. 5 ml of a bacterial solution was inoculated into 200 ml of an LB medium containing 50 μg/ml kanamycin sulfate and incubated at 37° C. and 220 rpm to OD=0.6 (about 3 h). Isopropyl-β-d-thiogalactoside (IPTG) was added to a final concentration of 1 mM, 16° C., 180 rpm for 16 h. The bacterial solution was transferred to a centrifuge tube, and centrifugation was conducted at 4° C., 4,000 rpm for 15 min. A supernatant was discarded, PBS was added to resuspend the precipitation at 4° C., 4,000 rpm for 15 min. A supernatant was discarded, 80 ml of the Binding Buffer was added to resuspend the bacteria, and the bacteria were solicited for rupture. A ruptured bacterial solution was centrifuged at 4° C., 12,000 rpm for 20 min, and the supernatant was collected. A nickel column was washed twice with ddH₂O and twice with the Binding Buffer, and the above supernatant was passed through the column at 6 s/drop. The column was washed 3 times with the Wash Buffer, and 20 ml of the Elution Buffer was added to elute a target protein. The eluted protein was added to a 3 KD ultrafiltration tube, and centrifugation was conducted at 4° C. and 4,000 rpm until 2 ml of liquid was remained. 8 ml of a PBS solution was added, and centrifugation was conducted at 4° C. and 4,000 rpm until 2 ml of liquid was remained. The steps were repeated 3 times. The protein in the ultrafiltration tube was aspirated, subjected to concentration measurement, and stored at 4° C.

2. Specificity Test with GAPDH Antibody

The flag antibody and the GAPDH antibody were subjected to doubling dilution, respectively, and detected using prokaryotic expression and purification NanoSwitch-1×flag-2NM.

3. 3×flag peptide competitive inhibition test

A3×flag polypeptide was synthesized, the 3×flag polypeptide was added to a NanoSwitch-1×flag-2NM +flag antibody reaction system at different concentrations, and changes in Nanoluc activity were detected. A 100 μg/ml irrelevant peptide p21 or pG4 was used as a control and added to the NanoSwitch-1×flag-2NM+flag antibody reaction system.

II. Results

1. Specificity Test with GAPDH Antibody

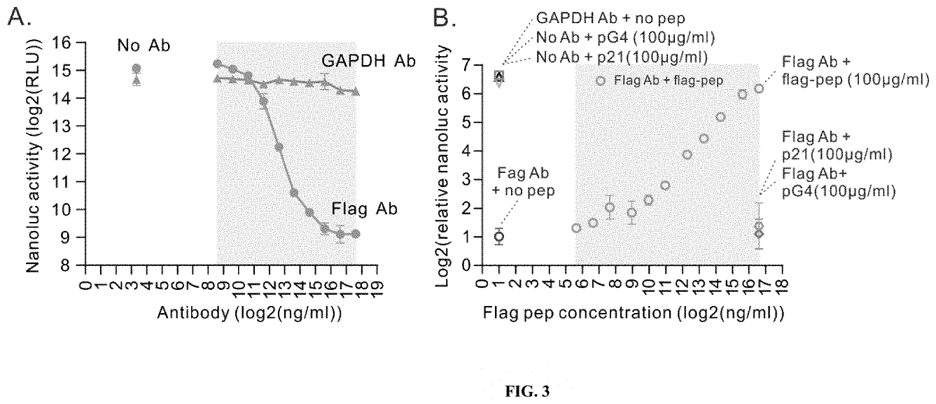

The results (FIG. 3A) show that, as the amount of flag antibody gradually increases, Nanoluc activity gradually decreases, while the increase in the amount of GAPDH antibody does not significantly change the Nanoluc activity.

The results show that the signal change of NanoSwitch-1×flag-2NM has a desirable specificity, and when the concentration of flag antibody is 1.6-51.2 μg/ml, there is a desirable dose-dependent relationship between the signal and the antibody concentration.

2. 3×flag peptide competitive inhibition test

The higher the concentration of 3×flag peptide (flag pep) is added, the Nanoluc activity of the reaction system will gradually recover due to competitive binding with the flag antibody. The control peptides p21 or pG4 cannot change the Nanoluc activity.

This result shows that: the binding of flag antibody to NanoSwitch-1×flag-2NM is specific, and this binding leads to the decrease of Nanoluc enzyme activity of NanoSwitch-1×flag-2NM, such that the signal change can reflect the amount of flag antibody.

In short, NanoSwitch-1×flag-2NM can specifically detect flag antibodies.

### Example 4 NanoSwitch for the Detection of SARS-CoV-2 Antibody

Two peptides, pG4 and p21, were placed in NanoSwitch in three different ways. A first way was only 1 copy at a C-terminus (labeled as pG4-C and p21-C); a second way was only 1 copy between LgBiT and SmBiT (labeled as pG4-M and p21-M); a third way was 1 copy between the LgBiT and the SmBiT, and 1 copy at an N-terminus (labeled as pG4-MC and p21-MC); a fourth way was a combination of the foregoing two, including two copied polypeptides (labeled as pG4-NM and p21-NM); and a fifth way was adding 1 copy at the N-terminus based on the third way (labeled as pG4-NMC and p21-NMC).

The pG4 (SEQ ID NO. 26) had a polypeptide sequence as follows:

LQPELDSFKEELDKYFKNHTSPDVD

The p21 (SEQ ID NO. 27) had a polypeptide sequence as follows:

PSKPSKRSFIEDLLFNKV

I. Method

A plasmid expressing the aforementioned NanoSwitch was synthesized using the methods of Examples 1 and 2. Compared with the plasmids obtained in Examples 1 and 2, the plasmid synthesized in this example only involved replacing a gene sequence of Flag or 3×Flag with a gene sequence of pG4 or p21, where a gene sequence of the pG4 (SEQ ID NO. 28) was as follows:

TTGCAACCTGAATTAGACTCATTCAAGGAGGAGTTAGATAAATATTTTAA
GAATCATACATCACCAGATGTTGAT

a gene sequence of the p21 (EQ ID NO. 29) was as follows:

CCATCAAAACCAAGCAAGAGGTCATTTATTGAAGATCTACTTTTCAACAA
AGTG

The plasmids were transfected into HEK293 cells, and 1 μl of serum from patients (4 patients) infected with COVID-19 was added to 9 μl of a diluted solution of lysate; meanwhile, sera from people infected with hepatitis B virus or healthy people were used as controls. After incubating for 20 min at room temperature, the Nanoluc activity was directly detected in the system.

II. Results

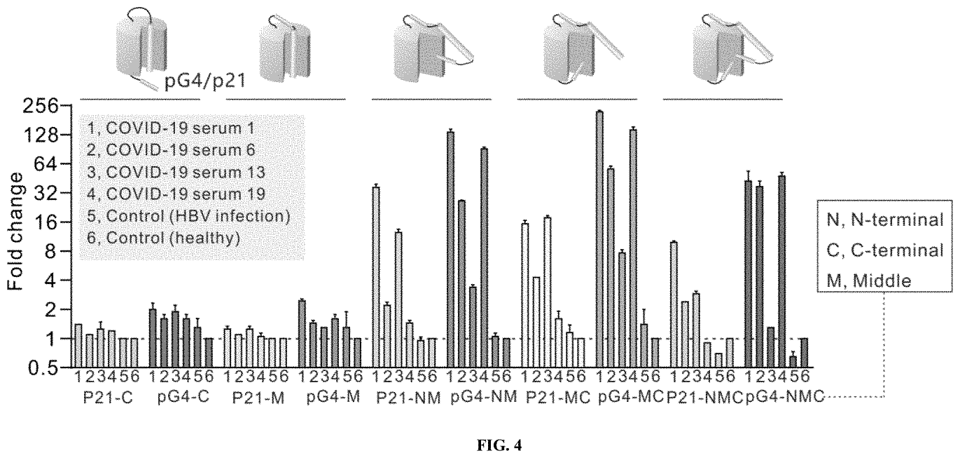

The results are shown in FIG. 4.

The two polypeptides cannot reflect the status of SARS-CoV-2 antibodies in the serum when using the first and second structures, that is, a single copy.

The third, fourth and fifth structures can reflect the situation of antibodies, where the fourth structure has a relatively higher signal-to-noise ratio. The molecular switches for detecting pG4 and p21 antibodies are named NanoSwitch-pG4 and NanoSwitch-p21, respectively. Compared between the two peptides, pG4 has a better detection effect than that of p21. For example, for sample No. 1, the signal of pG4-MC is increased by 223-fold more than that of healthy control serum.

It is worth noting that, unlike the case of NanoSwitch that detects flag antibodies in Examples 1 and 2, the NanoSwitch-pG4 and the NanoSwitch-p21 each show an increase in Nanoluc activity, not a decrease, after being added to the serum of patients with COVID-19.

The above results indicate that during the detection of pG4 antibody or p21 antibody, in addition to the antigen polypeptide between LgBiT and SmBiT, an antigen polypeptide is also required on the C-terminus side and/or the N-terminus side. Moreover, after the target antibody is detected, the luciferase activity increases rather than decreases.

### Example 5 Competitive Inhibition Test of NanoSwitch-pG4 and NanoSwitch-p21

A competitive inhibition test was conducted to prove the specificity of NanoSwitch-pG4 and NanoSwitch-p21 for the detection of SARS-CoV-2 antibodies. Two polypeptides, pG4 and p21 were synthesized. These two polypeptides were added to the NanoSwitch+SARS-CoV-2 serum system of the fourth structure in Example 4 at different concentrations, and the Nanoluc activity was detected.

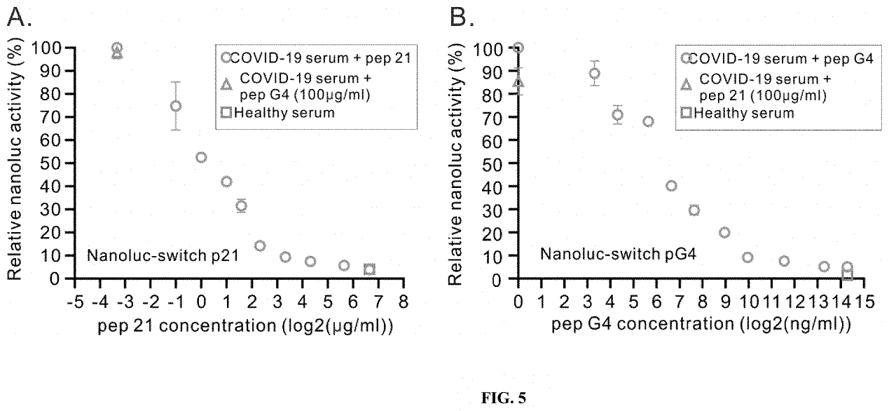

The results show that as the concentration of specific polypeptides increases, the Nanoluc activities of the two molecular switches gradually decrease (FIG. 5).

This indicates that the SARS-CoV-2 antibody in the serum can specifically bind to the above two NanoSwitch molecules.

### Example 6 Dynamic Range of NanoSwitch-pG4 for Detecting SARS-CoV-2 Antibodies

### I. Methods

Prokaryotic expression was conducted on NanoSwitch-pG4 with the fourth structure (that is, the middle and C-terminus had a pG4 polypeptide) in Example 4, gradient dilution was conducted on serum from patients with COVID-19 with a relatively high titer, and the linear range was detected.

A method of prokaryotic expression was as follows:

(1) Construction of plasmid PET-NanoSwitch-pG4

The plasmid PET28a was used as a template and amplification was conducted using primers F pet28a-Chis2+R rop-bsa1. A PCR reaction system was: 10 ng of the plasmid PET28a, 0.4 μl for each of the primers F pet28a-Chis2 (10 μM) and R rop-bsa1 (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 45 sec, conducting 35 cycles. An amplified fragment was recovered using the gel extraction kit, and the recovered fragment was named as frag19.

The plasmid PET28a was used as a template and amplification was conducted using primers R pet28a-Chis2+F rop-bsa1. A PCR reaction system was: 10 ng of the plasmid PET28a, 0.4 μl for each of the primers R pet28a-Chis2 (10 μM) and R rop-bsa1 (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 45 sec, conducting 35 cycles. An amplified fragment was recovered using the gel extraction kit, and the recovered fragment was named as frag20.

The plasmid SmBiT-LgBiT-2xpG4-MC was used as a template and amplification was conducted using primers F pet28a-cov+R pet28a-cov. A PCR reaction system was: 10 ng of the plasmid SmBiT-LgBiT-2xpG4-MC, 0.4 μl for each of the primers F pet28a-cov (10 μM) and R pet28a-cov (10 μM), 10 μl of a 2× PrimeSTAR Max Premix, and sterilized ultrapure water was added to make up a volume to 20 μl. The amplification reaction was conducted by: pre-denaturation at 95° C. for 3 min; denaturation at 95° C. for 15 sec, at 55° C. for 15 sec and at 72° C. for 20 sec, conducting 35 cycles. An amplified fragment was recovered using the gel extraction kit, and the recovered fragment was named as frag21.

The three fragments frag19, frag20 and frag21 obtained above were subjected to Golden Gate ligation reaction. A reaction system was: 0.75 μl of BsmB I enzyme, 1 μl of Tango buffer, 1 μl of DTT, 0.25 μl of T7 DNA ligase, 1 μl of ATP, 1 μl of frag19 (60 ng), 1 μl of frag20 (60 ng), 1 μl of frag21 (15 ng), and ddH₂O was added to make up to 10 μl. Reaction was conducted by: 37° C. for 4 min, 20° C. for 4 min, conducting 20 cycles. Inactivation reaction was conducted at 80° C. for 20 min. A Golden Gate product was transformed into JM109 competent bacteria, the bacteria were coated on plates, clones were screened, and sequenced for identification; a correct clone was named as a plasmid pET-NanoSwitch-pG4.

(2) Prokaryotic Expression and Purification of the NanoSwitch-pG4

A correctly sequenced plasmid pET-NanoSwitch-pG4 was transformed into Rosetta (DE3), spread on an LB plate containing 50 μg/ml kanamycin sulfate, and incubated at 37° C. for 16 h. A single colony was incubated in a 5 ml LB medium containing 50 μg/ml kanamycin sulfate at 37° C., 220 rpm for 16 h. 5 ml of a bacterial solution was inoculated into 200 ml of an LB medium containing 50 μg/ml kanamycin sulfate and incubated at 37° C. and 220 rpm to OD=0.6 (about 3 h). Isopropyl-β-d-thiogalactoside (IPTG) was added to a final concentration of 1 mM, 16° C., 180 rpm for 16 h. The bacterial solution was transferred to a centrifuge tube, and centrifugation was conducted at 4° C., 4,000 rpm for 15 min. A supernatant was discarded, PBS was added to resuspend at 4° C., 4,000 rpm for 15 min. A supernatant was discarded, 80 ml of the Binding Buffer was added to resuspend, and the bacteria were sonicated for rupture. A ruptured bacterial solution was centrifuged at 4° C., 12,000 rpm for 20 min, and a supernatant was collected. A nickel column was washed twice with ddH₂O and twice with the Binding Buffer, and the above supernatant was passed through the column at 6 s/drop. The column was washed 3 times with the Wash Buffer, and 20 ml of the Elution Buffer was added to elute a target protein. The eluted protein was added to a 3 KD ultrafiltration tube, and centrifugation was conducted at 4° C. and 4,000 rpm until 2 ml of liquid was remained. 8 ml of a PBS solution was added, and centrifugation was conducted at 4° C. and 4,000 rpm until 2 ml of liquid was remained. The steps were repeated 3 times. The protein in the ultrafiltration tube was aspirated, subjected to concentration measurement, and stored at 4° C.

2. Results

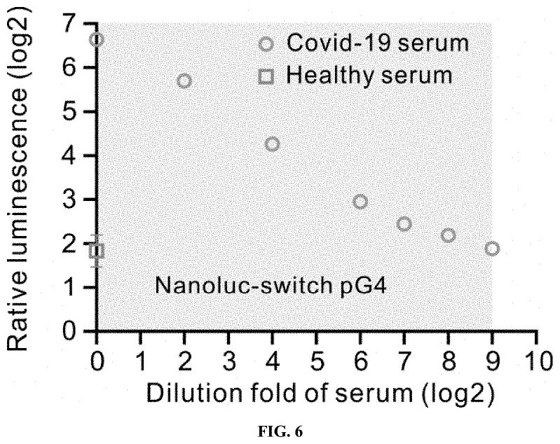

Before the sample is diluted to 512-fold, Nanoluc activity decreases as the dilution increases, showing a relatively wide linear range (FIG. 6).

### Example 7 NanoSwitch-pG4 in Detection of Serum Antibodies in Patients with SARS-CoV-2

I. Methods

The sera of 198 cases of non-COVID-19 patients and 111 positive sera of SARS-CoV-2 antibodies were detected using the NanoSwitch-pG4 obtained from prokaryotic expression in Example 6. The serum samples of these patients with COVID-19 were derived from a previous study, and a kit that had been approved for the clinical detection of SARS-CoV-2 antibodies (Antibody Responses to SARS-CoV-2 in Patients with COVID-19. Nature Medicine, 2020) was used to test these samples to determine that the samples were positive for the SARS-CoV-2 antibodies.

A detection process was as follows: 0.05 ng of prokaryotic expression and purification NanoSwitch-pG4 was dissolved in 9 μl of a buffer solution including: 50 mM Hepes (PH 7.5), 3 mM EDTA, 150 mM NaCl, 0.005% (v/v) Tween-20 and 10 mM DTT; 1 μl of serum was added to the above system, incubation was conducted at 37° C. for 10 min, and the sample was directly detected with a Nanoluc detection reagent (Promega).

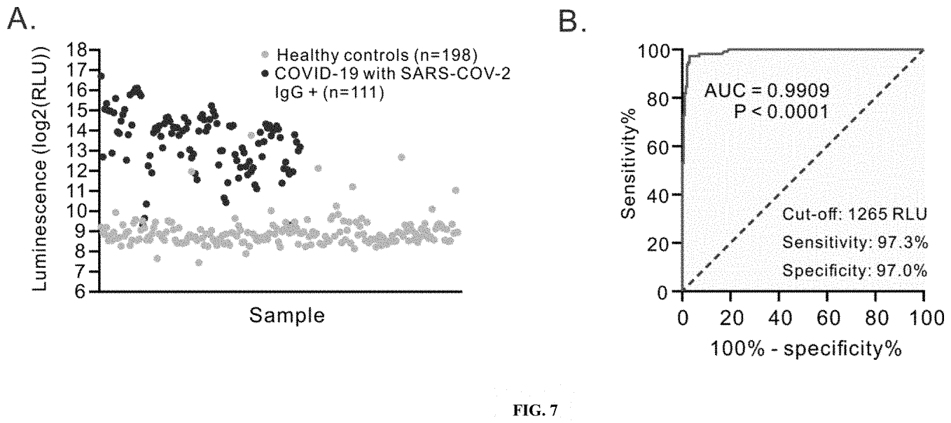

The detection results show that the NanoSwitch-pG4 can distinguish positive and negative SARS-CoV-2 antibodies well, with an area under the curve (AUC)=0.9909 in the receiver operator characteristic (ROC) (P <0.0001) (FIG. 7A). When a cutoff value is set at 1265, the sensitivity and specificity reach 97.3% and 97.0%, respectively (FIG. 7B).

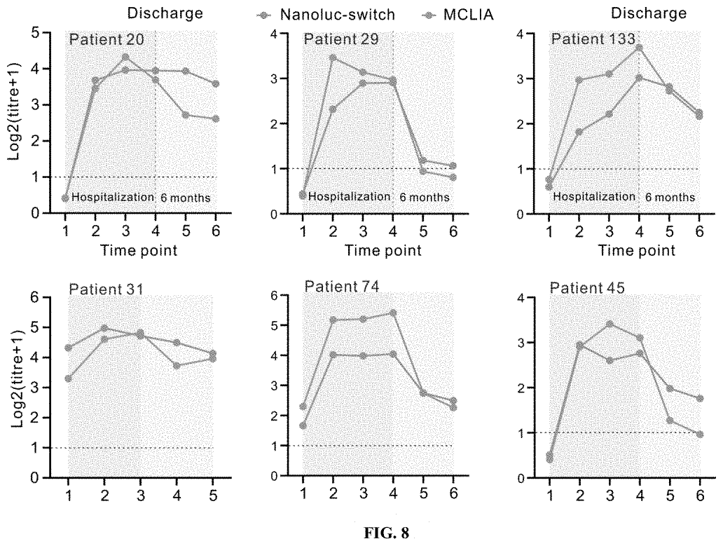

Based on this cutoff value, a SARS-CoV-2 antibody titer of each test sample was calculated, and an antibody titer change curve was drawn based on a test value of a serum sample from the same patient. It is found that the trend of antibody titer change measured according to this method is in desirable agreement with the trend of antibody titer change measured based on magnetic particle chemiluminescence immunoassay (MCLIA, approved for clinical testing). This can fully reflect the process of antibody changing from negative to positive in the early stage of infection, and the process of antibody rising or falling afterwards (FIG. 8 shows specific conditions of 6 patients). Compared with the MCLIA, this method is greatly simplified, including only three steps of sample loading, incubation and detection and without various washing and buffer replacement steps, which has a detection time of not more than 45 min. Meanwhile, only 1 μl of sample is required without dilution, which is very simple and convenient.

The results of this example show that the NanoSwitch-pG4 of the present disclosure can be used to detect SARS-CoV-2 antibodies with a simple process and high accuracy.

In summary, the molecular switch for antibody detection of the present disclosure can effectively detect various antibodies, with a large detection linear range, high specificity and high accuracy.

What is claimed is:

1. A protein-based multifunctional molecular switch for antibody detection, wherein the molecular switch is a fusion protein comprising the following parts ligated sequentially from an N-terminus to a C-terminus:
a part (1): SmBiT;
a part (2): an epitope polypeptide of an antibody to be detected; and
a part (3): LgBiT.

2. The molecular switch according to claim 1, wherein there is further an epitope polypeptide capable of being specifically bound by the antibody to be detected on an N-terminus side of the part (1) and/or a C-terminus side of the part (3).

3. The molecular switch according to claim 1, wherein there is a linker sequence between the parts (1) and (2).

4. The molecular switch according to claim 2, wherein there is a linker sequence between the parts (1) and (2).

5. The molecular switch according to claim 3, wherein the linker sequence is a flexible linker used for expressing the fusion protein, preferably a Gly-Ser (GS) linker.

6. The molecular switch according to claim 4, wherein the linker sequence is a flexible linker used for expressing the fusion protein, preferably a Gly-Ser (GS) linker.

7. The molecular switch according to claim 1, wherein the epitope polypeptide is a Flag tag;
preferably, there is a linker sequence between the parts (1) and (2);
more preferably, the linker sequence is a flexible linker used for expressing the fusion protein, furthermore preferably a GS linker.

8. The molecular switch according to claim 2, wherein the epitope polypeptide is a Flag tag;
preferably, there is a linker sequence between the parts (1) and (2);
more preferably, the linker sequence is a flexible linker used for expressing the fusion protein, furthermore preferably a GS linker.

9. The molecular switch according to claim 1, wherein the epitope polypeptide is a pG4 polypeptide;
preferably, there is a linker sequence between the parts (1) and (2);
more preferably, the linker sequence is a flexible linker used for expressing the fusion protein, furthermore preferably a GS linker.

10. The molecular switch according to claim 2, wherein the epitope polypeptide is a pG4 polypeptide;
preferably, there is a linker sequence between the parts (1) and (2);
more preferably, the linker sequence is a flexible linker used for expressing the fusion protein, furthermore preferably a GS linker.

11. The molecular switch according to claim 1, wherein the epitope polypeptide is a p21 polypeptide;
preferably, there is a linker sequence between the parts (1) and (2);
more preferably, the linker sequence is a flexible linker used for expressing the fusion protein, furthermore preferably a GS linker.

12. The molecular switch according to claim 2, wherein the epitope polypeptide is a p21 polypeptide;
preferably, there is a linker sequence between the parts (1) and (2);
more preferably, the linker sequence is a flexible linker used for expressing the fusion protein, furthermore preferably a GS linker.

13. A kit for detecting a 2019 novel coronavirus (SARS-CoV-2), comprising the molecular switch according to claim 9.

14. The kit for detecting a 2019 novel coronavirus (SARS-CoV-2) according to claim 13, wherein there is further an epitope polypeptide capable of being specifically bound by the antibody to be detected on an N-terminus side of the part (1) and/or a C-terminus side of the part (3).

15. A kit for detecting a 2019 novel coronavirus (SARS-CoV-2), comprising the molecular switch according to claim 11.

16. The kit for detecting a 2019 novel coronavirus (SARS-CoV-2) according to claim 15, wherein there is further an epitope polypeptide capable of being specifically bound by the antibody to be detected on an N-terminus side of the part (1) and/or a C-terminus side of the part (3).

17. The kit according to claim 13, further comprising a reagent for detecting a Nanoluc luciferase activity.

18. The kit according to claim 14, further comprising a reagent for detecting a Nanoluc luciferase activity.

19. The kit according to claim 15, further comprising a reagent for detecting a Nanoluc luciferase activity.

20. The kit according to claim 16, further comprising a reagent for detecting a Nanoluc luciferase activity.
